SEQUENCE LISTING (4 sequences)

SEQ ID NO: 1 - DNA, 20 bp, Artificial Sequence
  Forward primer for K-RAS
tgtcatcttgccctcctacc

SEQ ID NO: 2 - DNA, 19 bp, Artificial Sequence
  Reverse primer for K-RAS
tcaaagcatcagccaccac

SEQ ID NO: 3 - DNA, 20 bp, Artificial Sequence
  Forward primer for GAPDH
ccacggctgcttccagctcc

SEQ ID NO: 4 - DNA, 22 bp, Artificial Sequence
  Reverse primer for GAPDH
ggactccatgcccaggaaggaa

(19) United States
(12) Patent Application Publication
(10) Pub. No.: US 2020/0085805 A1
(43) Pub. Date: Mar. 19, 2020

(54) METHOD FOR TREATING CANCER RELATED TO ACTIVATION OF RAS GENE IN SUBJECT

(71) Applicant: Affiliated Hospital of Guilin Medical University, Guilin (CN)

(72) Inventors: Qinghua Li, Guilin (CN); Jingxin Mo, Guilin (CN); Rujia Liao, Guilin (CN); Zaiwa Wei, Guilin (CN); Liangxian Li, Guilin (CN); Xueyi Wen, Guilin (CN)

(21) Appl. No.: 16/245,230

(22) Filed: Jan. 10, 2019

(30) Foreign Application Priority Data
Sep. 14, 2018 (CN) 201811075013.X

(51) Int. Cl. A61K 31/4418; A61P 35/00

(52) U.S. Cl. CPC A61K 31/4418; A61P 35/00

(57) ABSTRACT

The invention relates to a method of treating a type of cancer related to activation of an RAS gene in a subject, and belongs to the field of pharmaceuticals. The method of treating cancer related to activation of an RAS gene involves administering to the subject a therapeutically effective amount of 1-methyl-4-phenyl-1,2,3,6-tetrahydropyridine, a pharmaceutically acceptable salt thereof, or a solvate thereof. 1-methyl-4-phenyl-1,2,3,6-tetrahydropyridine was found to reduce the level of RAS mRNA. 24 mM of 1-methyl-4-phenyl-1,2,3,6-tetrahydropyridine is capable of reducing RAS mRNA level by 3.5 folds. The compound of the present invention also has a significant tumor suppressive effect on a Drosophila tumor model. Clinical anti-tumor products that exploit the intervention and treatment of tumors by 1-methyl-4-phenyl-1,2,3,6-tetrahydropyridine have immense potential.

### CROSS-REFERENCE TO RELATED APPLICATIONS

The present application claims the benefit of Chinese Patent Application No. 201811075013.X filed on Sep. 14, 2018, the contents of which are hereby incorporated by reference.

### REFERENCE TO SEQUENCE LISTING

The Sequence Listing is submitted concurrently with the specification as an ASCII formatted text file via EFS-Web, with a file name of “Sequence_listing.txt”, a creation date of Jan. 10, 2019, and a size of 1,097 bytes. The Sequence Listing filed via EFS-Web is part of the specification and is incorporated in its entirety by reference herein.

### FIELD OF THE INVENTION

The invention relates to a method for treating a type of cancer related to activation of an RAS gene in a subject and belongs to the technical field of pharmaceuticals.

### BACKGROUND OF THE INVENTION

Cancer is a malignant disease with extremely high mortality and is difficult to treat. It brings heavy burdens to patients and families. In recent years, cancer incidence in China has increased significantly, which poses severe challenges to cancer prevention and control. The gradual increase in cancer incidence has attracted wide attention from society. According to researches, in the 1970s, cancer incidence in China increased from 10.13% to 22.32%, and the cancer mortality rate was increased by 82.11%. Cancer ranks first in mortality rate in urban areas and second in rural areas. An aging population, smoking, dietary changes, microbial infections, obesity, reduced physical activity, and irregular sleep patterns are among the main causes of cancer today. In China, in particular, overweight rate and obesity rate are significantly over 50%. At present, the top ten cancers in China are lung cancer, stomach cancer, colorectal cancer, liver cancer, esophageal cancer, female breast cancer, pancreatic cancer, lymphoma, bladder cancer, and thyroid cancer. Lung cancer is a common type of cancer in urban men; breast cancer is a common type of cancer in urban women. Stomach cancer is the most common cancer in rural men and women, and lung cancer has the highest mortality rate.

The RAS gene family includes three functional genes, namely H-RAS, N-RAS, and K-RAS. The nucleotide sequences of the three genes differ greatly, but all contain a non-coding exon at 5′ end and 4 coding exons. The encoded product of RAS genes is a monomeric G-protein with a relative molecular mass of 21,000 and is referred to as p21 protein (RAS protein). The RAS protein mainly regulates cell differentiation and proliferation, and is referred to as the “molecular switch” in cell signaling networks: when they are normal, they can control the pathway that regulates cell growth; when an abnormality occurs, the continuous growth of cells takes place, and the cells are prevented from self-destruction. The RAS genes are proto-oncogenes that, upon activation, become oncogenes with oncogenic activity. They are involved in intracellular signaling. When a K-RAS gene is mutated, it is permanently activated and is unable to produce normal RAS protein. This leads to disrupted intracellular signaling and uncontrolled cell proliferation, resulting in canceration. There are three main ways to activate a K-RAS gene, including point mutations, overexpression of genes, and gene insertion and gene transposition; the most common way is point mutation: about 30% of human malignancies have point mutations of the RAS genes. If a RAS protein is in an activated state continuously, it can bind to a downstream effector protein and transmit signals to downstream signaling elements, resulting in an abnormal proliferation of cells which leads to tumorigenesis.

Point mutation most frequently occurs in K-RAS, followed by N-RAS and H-RAS. Commonly mutated sites are codons 12, 13, and 61, with a mutation at codon 12 being the most common. Different RAS gene mutations occur in different types of tumors. K-RAS gene mutations are common in pancreatic cancer, colorectal cancer, endometrial cancer, cholangiocarcinoma, and lung cancer. Melanoma and myeloid malignancies are often caused by N-RAS gene mutations, whereas in liver cancer, thyroid cancer and bladder cancer, H-RAS gene mutations are more common. RAS genes are a kind of proto-oncogene that is evolutionarily conserved. They play an important role in a variety of cell-life activities, including cell proliferation and differentiation, and the construction of cytoskeleton. RAS gene proteins and their signaling pathways have been extensively studied, and it was found that RAS is closely related to the occurrence and progression of a tumor. Therefore, RAS protein has become a recognized target in the screening for anti-cancer drugs. This means that RAS inhibitors can be developed for tumor cell targeting.

The development of anti-cancer drugs has long been a hot spot in research and development. Chemical drugs and biologic drugs are in fierce competition, but new and effective malignancy-treating drugs are still in urgent demand.

### SUMMARY OF THE INVENTION

The objective of the present invention is to overcome the shortcomings of the prior art and to provide a method for treating a type of cancer related to activation of an RAS gene in a subject by administering to the subject a compound. The compound of the present invention reduces RAS mRNA level and has a significant tumor suppressive effect on a Drosophila tumor model. The compound of the present invention can be researched as an anticancer drug.

In order to achieve the objective above, the technical solution adopted by the present invention is: A method for treating a type of cancer related to activation of an RAS gene in a subject, comprising administering to the subject a therapeutically effective amount of a pyridine compound, a pharmaceutically acceptable salt thereof, or a solvate thereof, wherein the pyridine compound is a compound of Formula I, its IUPAC name is 1-methyl-4-phenyl-1,2,3,6-tetrahydropyridine (MPTP).

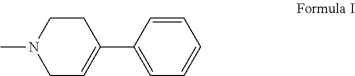

In a preferred embodiment, the RAS gene is activated by genetic mutation.

In a preferred embodiment, the RAS gene is selected from the group consisting of a K-RAS gene, an H-RAS gene, and an N-RAS gene.

In a preferred embodiment, the type of cancer includes glioblastoma multiforme, pancreatic cancer, colorectal cancer, endometrial cancer, cholangiocarcinoma, lung cancer, melanoma, myeloid cancer, liver cancer, thyroid cancer and bladder cancer.

The present invention evaluates the inhibitory effect of the compound on the amplification of RAS genes and the therapeutic effect of the compound on the Drosophila tumor model. It was found that the compound of the present invention can effectively reduce the level of RAS mRNA. 24 mM of 1-methyl-4-phenyl-1,2,3,6-tetrahydropyridine is capable of reducing RAS mRNA level by 3.5 folds and has excellent antitumor activities in Drosophila tumors. Therefore, clinical anti-tumor products that exploit the intervention and treatment of tumors by 1-methyl-4-phenyl-1,2,3,6-tetrahydropyridine have immense potential.

As a preferred embodiment of the method according to the present invention, the pharmaceutically acceptable salt is a pharmaceutically acceptable inorganic acid salt or a pharmaceutically acceptable organic acid salt formed from 1-methyl-4-phenyl-1,2,3,6-tetrahydropyridine and one or more than one kind of an inorganic acid or an organic acid.

As a preferred embodiment of the method according to the present invention, the pharmaceutically acceptable inorganic acid salt is selected from the group consisting of a hydrochloride salt, a hydrobromide salt, a phosphate salt, a sulfate salt, a perchlorate salt, and mixtures thereof.

As a preferred embodiment of the method according to the present invention, the pharmaceutically acceptable organic acid salt is selected from the group consisting of an acetate salt, an oxalate salt, a maleate salt, a tartrate salt, a citrate salt, a succinate salt, a malonate salt, and mixtures thereof.

As a preferred embodiment of the method according to the present invention, the pharmaceutically acceptable salt is selected from the group consisting of a dipate salt, an alginate salt, an ascorbate salt, an aspartate salt, a besylate salt, a benzoate salt, a bisulfate salt, a borate salt, a butyrate salt, a camphorate salt, a camphor sulfonate salt, a cyclopentylpropionate salt, a digluconate salt, a lauryl sulfate salt, an ethanesulfonate salt, a formate salt, a fumarate salt, a glucoheptonate salt, a glycerol phosphate salt, a gluconate salt, a hemisulfate salt, a heptanoate salt, a hexanoate salt, a hydroiodide salt, a 2-hydroxy-ethanesulfonate salt, a lactobionate salt, a lactate salt, a laurate salt, a lauryl sulfate salt, a malate salt, a methanesulfonate salt, a 2-naphthalenesulfonate salt, a nicotinate salt, a nitrate salt, a noleate salt, a palmitate salt, a pamoate salt, a pectate salt, a persulfate salt, a 3-phenylpropionate salt, a picrate salt, a pivalate salt, a propionate salt, a stearate salt, a thiocyanate salt, a p-toluenesulfonate salt, a nundecanoate salt, a valerate salt, and mixtures thereof.

As a preferred embodiment of the method according to the present invention, the pharmaceutically acceptable salt is selected from the group consisting of an alkali metal salt, an alkaline earth metal salt, an ammonium salt, a quaternary ammonium salt, and mixtures thereof. The alkali metal or alkaline earth metal include sodium, lithium, potassium, calcium, magnesium, among others. The ammonium salt is formed with an amine cation and a counterion, including a halide, a hydroxide, a carboxylate, a sulfate, a phosphate, a nitrate, a C₁₋₈ sulfonate, an aromatic sulfonate, among others.

As a preferred embodiment of the method according to the present invention, the solvate is an association of 1-methyl-4-phenyl-1,2,3,6-tetrahydropyridine with one or more solvent molecules.

As a preferred embodiment of the method according to the present invention, the solvent is selected from the group consisting of water, isopropanol, ethanol, methanol, dimethyl sulfoxide, ethyl acetate, acetic acid, or monoethanolamine, and mixtures thereof.

In a second aspect, the present invention provides a pharmaceutical composition for treating a type of cancer related to activation of an RAS gene in a subject, comprising 1-methyl-4-phenyl-1,2,3,6-tetrahydropyridine, a pharmaceutically acceptable salt thereof, a solvate thereof; and a pharmaceutically acceptable carrier. The pharmaceutically acceptable carrier includes a diluent, an excipient, a filler, a binder, a wetting agent, a disintegrating agent, an absorption enhancer, a surfactant, an adsorption carrier, a lubricant, or other conventional carriers in the field of pharmaceuticals. The medicament of the present invention can be prepared into various forms such as injections, tablets, powders, granules, capsules, oral liquids, ointments, and creams. The various dosage forms of the drug above can be prepared according to a conventional method in the field of pharmaceuticals.

In a third aspect, the present invention provides a method for inhibiting RAS gene expression in a subject.

Compared with the prior art, the present invention has the following beneficial effects: the compound of the present invention inhibits the level of RAS mRNA. 24 mM of 1-methyl-4-phenyl-1,2,3,6-tetrahydropyridine is capable of reducing RAS mRNA level by 3.5 folds. The compound of the present invention has a significant tumor suppressive effect on a Drosophila tumor model. Therefore, clinical anti-tumor products that exploit the intervention and treatment of tumors by 1-methyl-4-phenyl-1,2,3,6-tetrahydropyridine have immense potential.

### BRIEF DESCRIPTION OF THE DRAWINGS

FIG. 1 is a statistical diagram showing the inhibitory effect of the compound of the present invention on RAS mRNA expression.

FIG. 2 is an image showing the effect of the compound of the present application on the expression of Drosophila fluorescent protein (control group: upper figure; test group: lower figure).

FIG. 3 is a statistical histogram showing the expression level of the Drosophila fluorescent protein when treated with the compound of the present invention.

### DETAILED DESCRIPTION OF THE ILLUSTRATED EMBODIMENTS

In order to better illustrate the objectives, technical solutions and beneficial effects of the present invention, the present invention will be further described hereafter with reference to the accompanying drawings and detailed embodiments.

In the following embodiments, unless otherwise specified, the technical means employed are conventional means well known to those skilled in the art, and the reagents and materials in the present application can be obtained commercially or from other public sources.

### Embodiment 1

### Testing the Reduction of RAS mRNA by the Compound of the Present Invention

The molecular weight of 1-methyl-4-phenyl-1,2,3,6-tetrahydropyridine is 173.25 g/mol. 1.7325 g of the compound was accurately weighed, dissolved in 10 mL of DMSO solution to prepare a 1 M mother liquid for later use.

The preparation of a normal culture medium: 0.53 g of agar and 1.6 g of yeast extraction powder was added to 50 mL H₂O. The mixture obtained was heated until boiling. Then, 0.43 g of NaKT, 0.033 g of anhydrous CaCl₂, 1.58 g of sucrose, 3.17 g of glucose, and 3.88 g of corn flour (pre-mixed thoroughly with cold water to prevent agglomeration) were added to the mixture, and was brought to boil before stopping heating. The final mixture was transferred in aliquots into a number of culture tubes with a diameter of 25 mm and a volume of 5-8 mL. The filled culture tubes were sterilized at high temperature (30 min, 116° C.), followed by cooling to around 50° C. 1-2 drops of ampicillin stock solution were then added to the filled culture tubes, and the culture tubes were stored at 4° C. A small amount of active dry yeast was added before use. The ampicillin stock solution was prepared as follows: 8.2 mL of 95% ethanol and 7.4 mL of dH₂O were added to 0.0623 g of ampicillin, the mixture was homogenized by ultrasound and then stored at −20° C.

The preparation of a doped culture medium: an adequate amount of the mother liquid was added to the normal culture medium to prepare a culture medium doped with 24 mM of 1-methyl-4-phenyl-1,2,3,6-tetrahydropyridine.

In the present invention, the content of RAS mRNA was measured by RT-PCR. The total RNA of Drosophila was extracted according to instructions from a total RNA extraction kit (Tiangen Biotech Co., Ltd., Beijing). The extracted total RNA was reverse-transcribed to cDNA (by RNA prep pure Tissue Kit, Quantc DNA, and Quantone step qRT-PCR, which were all purchased from Thermo Co., Ltd., USA) , followed by amplification and analysis. GAPDH was used as an internal reference, and a K-RAS mRNA primer sequence was designed and synthesized by Shanghai Bioengineering Co., Ltd. The forward and reverse primers for K-RAS were respectively 5c-TGTCATCTTGCCCTCCTACC-3c (20 bp, SEQ ID NO.: 1) and 5c-TCAAAGCATCAGCCACCAC-3c (19 bp, SEQ ID NO.: 2). The forward and reverse primers for GAPDH were respectively 5c-CCACGGCTGCTTCCAGCTCC-3c (20 bp, SEQ ID NO.: 3) and 5c-GGACTCCATGCCCAGGAAGGAA-3c (22 bp, SEQ ID NO.: 4). The reaction conditions were a 25 μL reaction system, which includes predenaturation at 94° C. for 3 min, denaturation at 94° C. for 30 s, annealing at 52° C. for 30 s, elongation at 72° C. for 1 min, 35 cycles, final elongation at 72° C. for 5 min, and finally termination at 4° C. PCR amplification products corresponding to the K-RAS primers were 242 bp long respectively. PCR amplification products corresponding to the GAPDH primers were 295 bp long. After the amplification, the relative expression level of K-RAS in Drosophila was determined by the 2-ΔΔCt method.

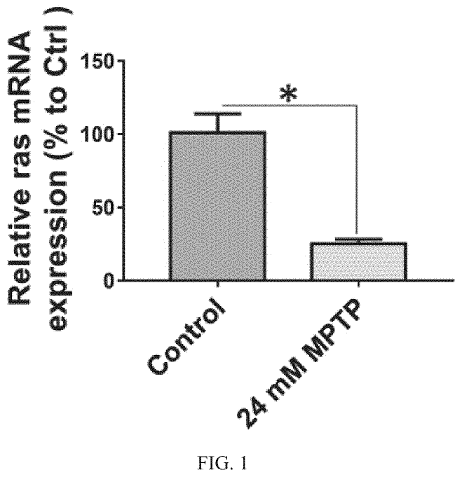

After culturing in the normal culture medium (as a control group) and a culture medium doped by 24 mM of 1-methyl-4-phenyl-1,2,3,6-tetrahydropyridine (as a test group) for 5 days, 40 pupae were taken from each of the culture tubes used for the test. The pupae were washed twice with PBS (phosphate buffered saline solution) and transferred to an ice box to freeze for 2 hours. The relative amount of RAS mRNA was determined by the above method. The test results are shown in FIG. 1.

As is apparent from FIG. 1, the compound of the present invention has an inhibitory effect on RAS protein. 24 mM of 1-methyl-4-phenyl-1,2,3,6-tetrahydropyridine can reduce RAS mRNA level by 3.5 folds.

### Embodiment 2

### Inhibition of Tumors in a Drosophila Tumor Model by the Compounds of the Present Invention

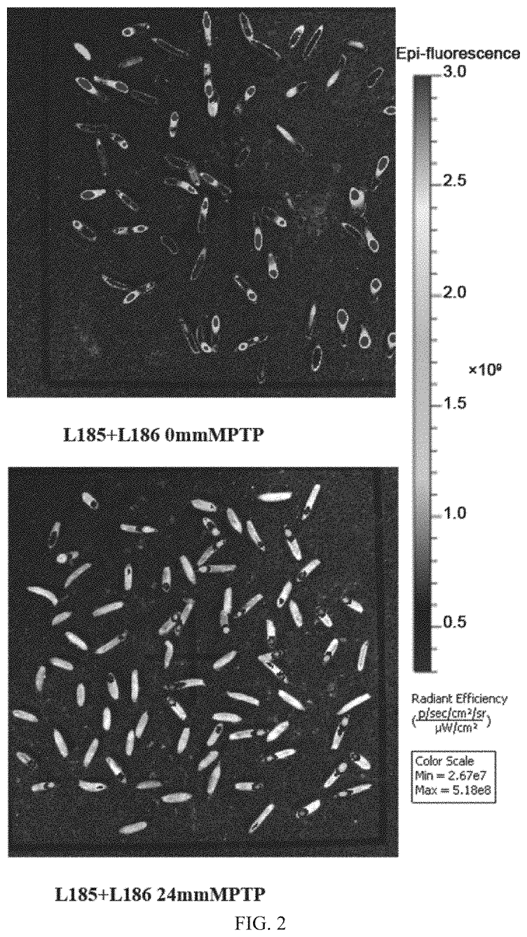

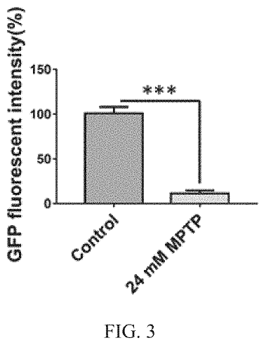

A green fluorescent protein (GFP)-tagged FLP/FRT recombinant system and scrib/TM6B balanced Drosophila melanogaster strain were used for hybridization to obtain a GFP-scrib−/− chimeric tumorigenesis model (L185+L186), wherein the GFP expression is in linkage with tumorigenesis. The preparation of a normal culture medium and a doped culture medium was the same as in embodiment 1. After culturing in the normal culture medium and the doped culture medium for 5 days, 80 pupae were taken from the culture tubes used for the test and washed twice with PBS (phosphate buffered saline solution). The pupae were neatly arranged on slides and were then transferred to an ice box to freeze for 2 hours. The pupae were observed under an IVIS Lumina X5 live imaging system (excitation light wavelength 450-490 nm, the same hereafter), images were taken, and the expression of the green fluorescent protein was evaluated. The results are shown in FIG. 2 and FIG. 3.

It can be understood from FIG. 2 and FIG. 3 that the compound of the present invention has good in vitro inhibitory activity against the L185+L186 Drosophila tumor model. When 1-methyl-4-phenyl-1,2,3,6-tetrahydropyridine was not used, the absolute value of the fluorescence of the Drosophila tumor model was 2.37×10⁹ μW/cm². After oral administration of 24 mM of 1-methyl-4-phenyl-1,2,3,6-tetrahydropyridine to the Drosophila tumor model, the absolute value of the fluorescence of the Drosophila tumor model was 3.5×10⁸ μW/cm², indicating that the compound of the present invention can be further studied as an anticancer drug.

It should be understood that the above embodiments are merely illustrative of the technical solutions of the present invention and are not intended to limit the scope of the present invention. Although the invention has been described in detail with reference to the preferred embodiments, those skilled in the art should understand that the technical solutions of the present invention may be modified or equivalently substituted without departing from the spirit and scope of the technical solutions of the present invention.

What is claimed is:

1. A method for treating a type of cancer related to activation of an RAS gene in a subject, comprising administering to the subject a therapeutically effective amount of a pyridine compound, a pharmaceutically acceptable salt thereof, or a solvate thereof, wherein the pyridine compound is a compound of Formula I, its IUPAC name is 1-methyl-4-phenyl-1,2,3,6-tetrahydropyridine;

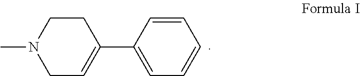

2. The method according to claim 1, wherein the RAS gene is activated by genetic mutation.

3. The method according to claim 1, wherein the RAS gene is selected from the group consisting of a K-RAS gene, an H-RAS gene, and an N-RAS gene.

4. The method according to claim 1, wherein the type of cancer includes glioblastoma multiforme, pancreatic cancer, colorectal cancer, endometrial cancer, cholangiocarcinoma, lung cancer, melanoma, myeloid cancer, liver cancer, thyroid cancer and bladder cancer.

5. The method according to claim 1, wherein the pharmaceutically acceptable salt is a pharmaceutically acceptable inorganic acid salt or a pharmaceutically acceptable organic acid salt formed from 1-methyl-4-phenyl-1,2,3,6-tetrahydropyridine and one or more than one kind of an inorganic acid or an organic acid.

6. The method according to claim 5, wherein the pharmaceutically acceptable inorganic acid salt is selected from the group consisting of a hydrochloride salt, a hydrobromide salt, a phosphate salt, a sulfate salt, a perchlorate salt, and mixtures thereof.

7. The method according to claim 5, wherein the pharmaceutically acceptable organic acid salt is selected from the group consisting of an acetate salt, an oxalate salt, a maleate salt, a tartrate salt, a citrate salt, a succinate salt, a malonate salt, and mixtures thereof.

8. The method according to claim 1, wherein the pharmaceutically acceptable salt is selected from the group consisting of an adipate salt, an alginate salt, an ascorbate salt, an aspartate salt, a besylate salt, a benzoate salt, a bisulfate salt, a borate salt, a butyrate salt, a camphorate salt, a camphorsulfonate salt, a cyclopentylpropionate salt, a digluconate salt, a lauryl sulfate salt, an ethanesulfonate salt, a formate salt, a fumarate salt, a glucoheptonate salt, a glycerol phosphate salt, a gluconate salt, a hemisulfate salt, a heptanoate salt, a hexanoate salt, a hydroiodide salt, a 2-hydroxy-ethanesulfonate salt, a lactobionate salt, a lactate salt, a laurate salt, a lauryl sulfate salt, a malate salt, a methanesulfonate salt, a 2-naphthalenesulfonate salt, a nicotinate salt, a nitrate salt, a noleate salt, a palmitate salt, a pamoate salt, a pectate salt, a persulfate salt, a 3-phenylpropionate salt, a picrate salt, a pivalate salt, a propionate salt, a stearate salt, a thiocyanate salt, a p-toluenesulfonate salt, an undecanoate salt, a valerate salt, and mixtures thereof.

9. The method according to claim 1, wherein the pharmaceutically acceptable salt is selected from the group consisting of an alkali metal salt, an alkaline earth metal salt, an ammonium salt, a quaternary ammonium salt, and mixtures thereof.

10. The method according to claim 1, wherein the solvate is an association of 1-methyl-4-phenyl-1,2,3,6-tetrahydropyridine with one or more solvent molecules.

11. The method according to claim 10, wherein the solvent molecule is selected from the group consisting of water, isopropanol, ethanol, methanol, dimethyl sulfoxide, ethyl acetate, acetic acid, monoethanolamine, and mixtures thereof.

12. A pharmaceutical composition for treating a type of cancer related to activation of an RAS gene in a subject, wherein the pharmaceutical composition comprises 1-methyl-4-phenyl-1,2,3,6-tetrahydropyridine, a pharmaceutically acceptable salt thereof, or a solvate thereof; and a pharmaceutically acceptable carrier.

13. The pharmaceutical composition according to claim 12, wherein the pharmaceutically acceptable salt is a pharmaceutically acceptable inorganic acid salt or a pharmaceutically acceptable organic acid salt formed from 1-methyl-4-phenyl-1,2,3,6-tetrahydropyridine and one kind or more than two kinds of an inorganic acid or an organic acid.

14. The pharmaceutical composition according to claim 13, wherein the pharmaceutically acceptable inorganic acid salt is selected from the group consisting of a hydrochloride salt, a hydrobromide salt, a phosphate salt, a sulfate salt, a perchlorate salt, and mixtures thereof.

15. The pharmaceutical composition according to claim 13, wherein the pharmaceutically acceptable organic acid salt is selected from the group consisting of an acetate salt, an oxalate salt, a maleate salt, a tartrate salt, a citrate salt, a succinate salt, a malonate salt, and mixtures thereof.

16. The pharmaceutical composition according to claim 12, wherein the pharmaceutically acceptable salt is selected from the group consisting of an adipate salt, an alginate salt, an ascorbate salt, an aspartate salt, a besylate salt, a benzoate salt, a bisulfate salt, a borate salt, a butyrate salt, a camphorate salt, a camphorsulfonate salt, a cyclopentylpropionate salt, a digluconate salt, a lauryl sulfate salt, an ethanesulfonate salt, a formate salt, a fumarate salt, a glucoheptonate salt, a glycerol phosphate salt, a gluconate salt, a hemisulfate salt, a heptanoate salt, a hexanoate salt, a hydroiodide salt, a 2-hydroxy-ethanesulfonate salt, a lactobionate salt, a lactate salt, a laurate salt, a lauryl sulfate salt, a malate salt, a methanesulfonate salt, a 2-naphthalenesulfonate salt, a nicotinate salt, a nitrate salt, a noleate salt, a palmitate salt, a pamoate salt, a pectate salt, a persulfate salt, a 3-phenylpropionate salt, a picrate salt, a pivalate salt, a propionate salt, a stearate salt, a thiocyanate salt, a p-toluenesulfonate salt, an undecanoate salt, a valerate salt, and mixtures thereof.

17. The pharmaceutical composition according to claim 12, wherein the pharmaceutically acceptable salt is selected from the group consisting of an alkali metal salt, an alkaline earth metal salt, an ammonium salt, a quaternary ammonium salt, and mixtures thereof.

18. The pharmaceutical composition according to claim 12, wherein the solvate is an association of 1-methyl-4-phenyl-1,2,3,6-tetrahydropyridine with one or more solvent molecules.

19. The pharmaceutical composition according to claim 18, wherein the solvent molecule is at least one of water, isopropanol, ethanol, methanol, dimethyl sulfoxide, ethyl acetate, acetic acid, or monoethanolamine.

20. A method for inhibiting RSA gene expression in a subject, comprising administrating to the subject an effective amount of a pyridine compound, a pharmaceutically acceptable salt thereof, or a solvate thereof, wherein the pyridine compound is a compound of Formula I, its IUPAC name is 1-methyl-4-phenyl-1,2,3,6-tetrahydropyridine

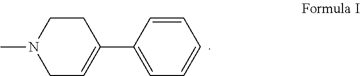

21. The method for inhibiting RSA gene expression in the subject according to claim 20, comprising inhibiting RNA mRNA.
